(19) United States
(12) Patent Application Publication
(10) Pub. No.: US 2004/0110166 A1
(43) Pub. Date: Jun. 10, 2004

(54) Genome-wide scanning of genetic polymorphisms

(76) Inventor: Stephen C. Macevicz, Cupertino, CA (US)

Correspondence Address: Stephen C. Macevicz, 21890 Rucker Drive, Cupertino, CA, 95014, US

(21) Appl. No.: 10/383,371

(22) Filed: Mar. 6, 2003

Related U.S. Application Data
(60) Provisional application No. 60/362,666, filed on Mar. 7, 2002.

(51) Int. Cl. C12Q 1/68; C12M 1/34

(52) U.S. Cl. 435/6; 435/287.1

(57) ABSTRACT

A method and compositions are provided for analyzing whole genomes, or representations thereof, to determine associations between traits and genotypes. Sets of hybridization probes (referred to herein as “isostringency probes”) are provided that are complementary to sites uniformly spaced throughout unique sequence regions a genome, or target polynucleotide, and that are designed for facile isolation of subsets that form perfectly matched duplexes with the genome or target polynucleotide being analyzed. The nucleotide sequences of the isostringency probes are selected to ensure that the probes have substantially identical duplex stabilities. In accordance with tie method of the invention, representations of a genome are attached to solid phase supports and are used to capture isostringency probes forming perfectly matched duplexes. The captured probes are then released and applied to an array of complementary sequences for detection.

### FIELD OF THE INVENTION

The invention relates generally to compositions and methods for genetic analysis, and more particularly, to hybridization-based methods for detecting polymorphisms throughout a genome or a population of genomes.

### BACKGROUND

Unraveling the genetic basis of complex traits remains an unsolved problem of immense medical and economic importance. One approach to this problem is to carry out trait-association studies in which a large set of genetic markers from populations of affected and unaffected individuals are compared. Such studies depend on the non-random segregation, or linkage disequilibrium, between the genetic markers and genes involved in the trait or disease being studied. Unfortunately, the extent and distribution of linkage disequilibrium between regions of the human genome is not well understood, but it is currently believed that successful trait-association studies in humans would require the measurement of 30-50,000 markers per individual in populations of at least 300-400 affected individuals and an equal number of controls, Kruglyak and Nickerson Nature Genetics, 27: 234-236 (2001); Lai, Genome Research, 11: 927-929 (2001); Risch and Merikangas, Science, 273: 1516-1517 (1996); Cardon and Bell, Nature Reviews Genetics, 2: 91-99 (2001). The cost of such studies using current technology is staggering, Weaver, Trends in Genetics, pgs. 36-41 (December, 2000).

Single nucleotide polymorphisms (SNPs) are the markers of choice for trait-association studies, because their density (about 1 per 1000 nucleotides in mammalian genomes) may be high enough to permit determination of trait-causing genes by linkage disequilibrium between such genes and nearby SNP markers, Wang et al, Science, 280: 1077-1082 (1998). So-called common SNPs (present at a frequency of>20% in a population and appearing at a rate of about 1 per 1000 basepairs in human populations) are of special interest because statistically meaningful measurements of association can be obtained from smaller sample and control populations, Lai (cited above), Weiss and Clark, Trends in Genetics, 18: 19-24 (2002). However, for a given population being studied, it is difficult to know ahead of time which SNPs are common and which are not without carrying out extensive measurements. This has led at least one geneticist to suggest the need for a technique capable of simultaneously detecting and measuring SNPs. Lai (cited above).

All current genotyping methodologies, save invasive cleavage^{x}, require an amplification step that is usually implemented by a polymerase chain reaction (PCR), Gut, Human Mutation, 17: 475-492 (2001). In some cases, SNPs at multiple loci can be simultaneously measured in a multiplexed PCR, where up to several tens of amplifications are carried out in the same reaction mixture. However, appropriate amplification of multiple loci is difficult to achieve in this approach without extraordinary care in the selection of primers and reaction conditions. Moreover, even with successful multiplexing, the number of reactions required for a modest-sized trait-association study is still astronomical. Pooling of DNA from multiple samples has also been used to reduce the total number of amplification is required for association studies, e.g. Barcellos et al, Am. J. Hum. Genet., 61: 734-747 (1997), Breen et al, Biotechniques, 28: 464-470 (2000), Germer et al, Genome Research, 10: 258-266 (2000). In this approach, a single locus is amplified from DNA pooled from multiple individuals. Typically, the locus contains a polymorphic microsatellite marker or a restriction fragment length polymorphism and a readout of allele frequencies is obtained by densimetric analysis of electrophoretically separated DNA fragment. The process is highly labor intensive when applied to a large number of loci. More recently, pooling also has been suggested for measuring SNP frequencies at multiple loci in a “mini-sequencing” protocol, Fan et al, Genome Research, 10: 853-860 (2000). In this approach, SNPs are detected by extending primers conjugated to unique oligonucleotide tags, wherein each tag corresponds to a different genetic locus. After extension with labeled dideoxynucleoside triphosphates, the conjugates are specifically hybridized to a microarray of tag complements where SNP frequencies are readout as differentially labeled elements in the microarray. This method, like others, requires prior determination of common SNPs, the nucleotide sequences of the respective genetic loci, a separate amplification for each locus, and the synthesis a corresponding primer-tag conjugate for each locus.

In view of the above, the fields of medical and industrial genetics would be advanced by the availability of a technique to carry out accurately and economically trait-association studies without the need for large numbers of separate amplification reactions for each, or every few, genetic loci analyzed. In particular, the ability to survey thousands of genetic loci uniformly spaced over a whole genome in hundreds of individuals in a cost-effective manner would open the door to understanding the genetic basis of complex traits.

### SUMMARY OF THE INVENTION

Accordingly, objects of the invention include, but are not limited to, providing a method and compositions for analyzing whole genomes, or representations thereof, in order to determine associations between traits and genotypes; providing a method and compositions for parallel analysis of many thousands of genetic polymorphisms uniformly spaced over a genome; providing sets of genes and polymorphisms associated with specific amplicons; and providing a method and compositions for large scale genotyping of sequenced genomes.

The invention achieves these and other objectives in its various aspects and embodiments by the construction and use of sets of hybridization probes (referred to herein as “isostringency probes”) that are complementary to sites uniformly spaced throughout a genome, or target polynucleotide, and are designed for facile isolation of subsets that form perfectly matched duplexes with the genome or target polynucleotide being analyzed. The nucleotide sequences of the isostringency probes are selected to ensure that the probes have substantially identical duplex stabilities.

Preferably, the method of the invention is carried out with the following steps: (a) amplifying a population of restriction fragments from a genome, or a plurality of genomes, to form an amplicon; (b) capturing the amplicon on one or more solid phase capture supports; (c) hybridizing a plurality of isostringency probes to the captured amplicons; (d) isolating from the captured amplicon isostringency probes that form perfectly matched duplexes with the captured amplicon; and (e) specifically hybridizing the perfectly matched isostringency probes with their respective complements on one or more solid phase detection supports. Preferably, a reference sequence is available for the genome or target polynucleotide being analyzed for variation.

Nucleotide sequences of isostringency probes may or may not be selected so that they are complementary to regions containing known sequence polymorphisms, such as a known common SNP. In the preferred embodiment of the invention, sequence polymorphisms, i.e. deviations from the reference sequence, are detected by the failure of an isostringency probe to form a perfectly matched duplex at its complementary site in a test genome. Thus, probes may be selected to detect the presence or absence of a known polymorphism, or they may be selected without reference to a known polymorphism. In the latter case, isostringency probe hybridization is used to interrogate the nucleotides in its binding site in order to detect the presence or absence of polymorphism at any nucleotide position in the site. A failure of an isostringency probe to form a perfectly matched duplex indicates the presence of a sequence polymorphism in the test polynucleotide, e.g. due to a SNP in the binding site, or due to a SNP in an adjacent restriction site which eliminates the restriction fragment containing the binding site. The location of a polymorphism is determined from a probe's sequence and the locations of the amplified restriction fragments.

Preferably, the sequences of isostringency probes are selected so that their complementary sequences are in unique sequence regions of the genome or target polynucleotide being analyzed.

In one aspect, the invention provides a method and compositions for measuring polymorphisms in a set of genes or gene fragments located in a particular amplicon, such as an amplicon produced by amplification of NotI-MseI fragments of a genome, or a subset thereof selected by AFLP, or a similar technique. In this aspect, sets of genes may be grouped according to presence or absence in a predetermined amplicon and examined in parallel by specific sets of isostringency probes. In particular, the invention includes kits comprising sets of probes and/or isostringency probes to a plurality of genes contained in a predetermined amplicon.

Another aspect of the invention is the comparison of genomes or sets of genomes associated with different traits of interest, such as susceptibility to a disease. In this aspect, the method of the invention is preferably carried out by the following steps, illustrated in FIGS. 1 and 2: (a) amplifying equivalent sets of restriction fragments from genomes associated with a reference population and a test population to form an amplicon for each population; (b) separately capturing each amplicon on one or more solid phase capture supports; (c) hybridizing isostringency probes to each of the captured amplicons, such that isostringency probes hybridized to different amplicons have distinguishable labels; (d) isolating from each captured amplicon isostingency probes that form perfectly matched duplexes with the captured amplicons; and (e) specifically hybridizing the perfectly matched isostringency probes with their respective complements on one or more solid phase detection supports, such as a microarray, so that the isostringency probes at each hybridization site, or address, generate a signal indicative of the relative frequency of their respective complements in the populations. As used herein, “equivalent sets” in reference to restriction fragments means that the different populations of genomes, or target polynucleotides, are treated identically with whatever restriction enzymes that are used in an embodiment of the method.

In another aspect, the invention includes sets of isostringency probes for carrying out the method of the invention for particular genomes or subsets of particular genomes. Preferably, such sets of isostringency probes are components of a kit for carrying out the method of the invention. Usually, kits of the invention comprise isostringency probes and one or more solid phase detection supports. More preferably, kits of the invention comprise isostringency probes, one or more solid phase detection supports, and one or more solid phase capture supports. Still more preferably, such kits further include amplification adaptors, primers, and one or more restriction endonucleases.

The present invention overcomes shortcomings in the art by providing a method and materials for measuring frequencies of large numbers of genetic markers at thousands of loci uniformly spaced over a genome. The invention obviates the need for individual amplifications at each loci analyzed by amplifying sets of restriction fragments using adaptors containing primer binding sites. Multiple loci on the amplified fragments are simultaneously scanned by hybridization of predetermined sets of isostringency probes, preferably constructed from a reference genome sequence. The failure of isostringency probes to form perfectly matched duplexes with test sequences provides a measure of the presence of sequence polymorphisms. By comparing pools of genomes from control or reference populations and diseased populations isostringency probes, associations can be established between the frequencies of sequence markers in the control population and those of the disease population.

### BRIEF DESCRIPTION OF THE DRAWINGS

FIG. 1 illustrates an embodiment of the invention wherein two genomes are compared by hybridization of isostringency probes.

FIG. 2 illustrates how isostringency probes provide information about the presence of sequence polymorphism.

FIG. 3 illustrates an embodiment of the invention wherein relative frequencies of sequence markers in two populations are compared by competitive hybridization of isostringency probes to a microarray.

FIG. 4 illustrates an embodiment of the invention wherein relative frequencies of sequence markers are measured in a genome by hybridization of isostringency probes.

### DEFINITIONS

“Complement” or “tag complement” as used herein in reference to oligonucleotide tags refers to an oligonucleotide to which an oligonucleotide tag specifically hybridizes to form a perfectly matched duplex or triplex. In embodiments where specific hybridization results in a triplex, the oligonucleotide tag may be selected to be either double stranded or single stranded. Thus, where triplexes are formed, the term “complement” is meant to encompass either a double stranded complement of a single stranded oligonucleotide tag or a single stranded complement of a double stranded oligonucleotide tag.

The term “oligonucleotide” as used herein includes linear oligomers of natural or modified monomers or linkages, including deoxyribonucleosides, ribonucleosides, anomeric forms thereof, peptide nucleic acids (PNAs), and the like, capable of specifically binding to a target polynucleotide by way of a regular pattern of monomer-to-monomer interactions, such as Watson-Crick type of base pairing, base stacking, Hoogsteen or reverse Hoogsteen types of base pairing, or the like. Usually monomers are linked by phosphodiester bonds or analogs thereof to form oligonucleotides ranging in size from a few monomeric units, e.g. 3-4, to several tens of monomeric units, e.g. 40-60. Whenever an oligonucleotide is represented by a sequence of letters, such as “ATGCCTG,” it will be understood that the nucleotides are in 5′→3′ order from left to right and that “A” denotes deoxyadenosine, “C” denotes deoxycytidine, “G” denotes deoxyguanosine, and “T” denotes thymidine, unless otherwise noted. Usually oligonucleotides of the invention comprise the four natural nucleotides; however, they may also comprise non-natural nucleotide analogs. It is clear to those skilled in the art when oligonucleotides having natural or non-natural nucleotides may be employed, e.g. where processing by enzymes is called for, usually oligonucleotides consisting of natural nucleotides are required.

“Perfectly matched” in reference to a duplex means that the poly- or oligonucleotide strands making up the duplex form a double stranded structure with one other such that every nucleotide in each strand undergoes Watson-Crick basepairing with a nucleotide in the other strand. The term also comprehends the pairing of nucleoside analogs, such as deoxyinosine, nucleosides with 2-aminopurine bases, and the like, that may be employed. In reference to a triplex, the term means that the triplex consists of a perfectly matched duplex and a third strand in which every nucleotide undergoes Hoogsteen or reverse Hoogsteen association with a basepair of the perfectly matched duplex. Conversely, a “mismatch” in a duplex between a tag and an oligonucleotide means that a pair or triplet of nucleotides in the duplex or triplex fails to undergo Watson-Crick and/or Hoogsteen and/or reverse Hoogsteen bonding.

As used herein, “nucleoside” includes the natural nucleosides, including 2′-deoxy and 2′-hydroxyl forms, e.g. as described in Kornberg and Baker, DNA Replication, 2nd Ed. (Freeman, San Francisco, 1992). “Analogs” in reference to nucleosides includes synthetic nucleosides having modified base moieties and/or modified sugar moieties, e.g. described by Scheit, Nucleotide Analogs (John Wiley, New York, 1980); Uhlman and Peyman, Chemical Reviews, 90: 543-584 (1990), or the like, with the only proviso that they are capable of specific hybridization. Such analogs include synthetic nucleosides designed to enhance binding properties, reduce complexity, increase specificity, and the like.

As used herein “sequence determination” or “determining a nucleotide sequence” in reference to polynucleotides includes determination of partial as well as full sequence information of the polynucleotide. That is, the term includes sequence comparisons, fingerprinting, and like levels of information about a target polynucleotide, as well as the express identification and ordering of nucleosides, usually each nucleoside, in a target polynucleotide. The term also includes the determination of the identity, ordering, and locations of one, two, or three of the four types of nucleotides within a target polynucleotide. For example, in some embodiments sequence determination may be effected by identifying the ordering and locations of a single type of nucleotide, e.g. cytosines, within the target polynucleotide “CATCGC . . . ” so that its sequence is represented as a binary code. e .g. “100101 . . . ” for “C-(not C)-(not C)-C-(not C)-C . . . ” and the like.

As used herein “signature sequence” means a sequence of nucleotides derived from a polynucleotide such that the ordering of nucleotides in the signature is the same as their ordering in) the polynucleotide and the sequence contains sufficient information to identify the polynucleotide in a population. Signature sequences may consist of a segment of consecutive nucleotides (such as, (a,c,g,t,c) of the polynucleotide “acgtcggaaatc”), or it may consist of a sequence of every second nucleotide (such as, (c,t,g,a,a,) of the polynucleotide “acgtcggaaatc”), or it may consist of a sequence of nucleotide changes (such as, (a,c,g,t,c,g,a,t,c) of the polynucleotide “acgtcggaaatc”), or like sequences.

As used herein, the term “complexity” in reference to a population of polynucleotides means the number of different species of polynucleotide present in the population.

As used herein, “amplicon” means the product of an amplification reaction. That is, it is a population of polynucleotides, usually double stranded, that are replicated from one or more starting sequences. The one or more starting sequences may be one or more copies of the same sequence, or it may be a mixture of different sequences. Preferably, amplicons are produced either in a polymerase chain reaction (PCR) or by replication in a cloning vector.

As used herein, “addressable” in reference to tag complements means that the nucleotide sequence, or perhaps other physical or chemical characteristics, of a tag complement can be determined from its address, i.e. a one-to-one correspondence between the sequence or other property of the tag complement and a spatial location on, or characteristic of, the solid phase support to which it is attached. Preferably, an address of a tag complement is a spatial location, e.g. the planar coordinates of a particular region containing copies of the tag complement. However, tag complements may be addressed in other ways too, e.g. by microparticle size, shape, color, frequency of micro-transponder, or the like, e.g. Chandler et al, PCT publication WO 97/14028.

As used herein, “ligation” means to form a covalent bond or linkage between the termini of two or more nucleic acids, e.g. oligonucleotides and/or polynucleotides, in a template-driven reaction. The nature of the bond or linkage may vary widely and the ligation may be carried out enzymatically or chemically. As used herein, ligations are usually carried out enzymatically.

As used herein, “microarray” refers to a solid phase support, which may be planar or a collection of microparticles, that carries or carry oligo- or polynucleotides fixed or immobilized, usually covalently, at specific addressable locations. Preferably, a microarray is a solid phase support having a planar surface, which carries an array of nucleic acids, each member of the array comprising identical copies of an oligonucleotide or polynucleotide immobilized to a fixed region, which does not overlap with those of other members of the array. Typically, the oligonucleotides or polynucleotides are single stranded and are covalently attached to the solid phase support at known, determinable, or addressable, locations. The density of non-overlapping regions containing nucleic acids in a microarray is typically greater than 100 per cm², and more preferably, greater than 1000 per cm². Microarray technology is reviewed in the following references: Schena, Editor, Microarrays: A Practical Approach (YRL Press, Oxford, 2000); Southern, Current Opin. Chem. Biol., 2: 404-410 (1998); Nature Genetics Supplement, 21: 1-60 (1999).

As used herein, “genetic locus,” or “locus” in reference to a genome or target polynucleotide, means a contiguous subregion or segment of the genome or target polynucleotide. As used herein, genetic locus, or locus, may refer to the position of a gene or portion of a gene in a genome, or it may refer to any contiguous portion of genomic sequence whether or not it is within, or associated with, a gene. Preferably, a genetic locus refers to any portion of genomic sequence from a few tens of nucleotides, e.g. 10-30, in length to a few hundred nucleotides, e.g. 100-300, in length.

As used herein, “sequence marker” means a portion of nucleotide sequence at a genetic locus. A sequence marker may or may not contain one or more single nucleotide polymorphisms, or other types of sequence variation, relative to a reference or control sequence. In accordance with the invention, a sequence marker may be interrogated by specific hybridization of an isostringency probe.

As used herein, “allele frequency” in reference to a genetic locus, a sequence marker, or the site of a nucleotide means the frequency of occurrence of a sequence or nucleotide at such genetic loci or the frequency of occurrence of such sequence marker, with respect to a population of individuals. In some contexts, an allele frequency may also refer to the frequency of sequences not identical to, or exactly complementary to, a reference sequence.

As used herein, “uniform” in reference to spacing or distribution means that a spacing between objects, such as sequence markers, or events may be approximated by an exponential random variable, e.g. Ross, Introduction to Probability Models, 7^{th} edition (Academic Press, New York, 2000). In regard to spacing of sequence markers in a mammalian genome, it is understood that there are significant regions of repetitive sequence DNA in which a random sequence model of the genomic DNA does not hold. “Uniform” in reference to spacing of sequence markers preferably refers to spacing in uniques sequence regions, i.e. non-repetitive sequence regions, of a genome.

### DETAILED DESCRIPTION OF THE INVENTION

The invention addresses the problem of determining the frequency of sequence markers, such as single nucleotide polymorphisms (SNPs), at large numbers of genetic loci in one or more genomes without the need of separate amplifications for each loci of each genome. In particular, the invention provides a method for measuring simultaneously the relative frequencies of sequence markers at a plurality of genetic loci in control, or reference, populations and test populations. Thus, the invention permits the identification of regions of a genome that may contain genes or other genetic features associated with, and possibly responsible for, complex traits.

A key feature of the invention is the use of hybridization probes having substantially identical duplex stability, as determined by conventional measures, such as melting temperature, dissociation temperature, or the like. Such hybridization probes are referred to herein as “isostringency probes.”Preferably, sets of isostringency probes are formed for which hybridization and wash conditions can be selected that permit substantially all the probes forming perfectly matched duplexes with a target polynucleotide to be isolated, e.g. by dissociation by a small change in the stringency of the hybridization and/or wash conditions. Preferably, probe sequences within a set and wash conditions are selected so that “substantially all” means at least seventy percent of the probes eluted from an amplicon are perfectly matched probes; and more preferably, at least eighty percent; still more preferably, at least ninety percent; and most preferably, at least ninety-eight percent. Preferably, such a change in stringency is accomplished raising the reaction temperature by an amount less than or equal to about 5° C. As used herein, “stringency” refers to hybridization and/or wash conditions that tend to promote the maintenance of only perfectly matched duplexes in a hybridization or wash reaction. Preferably, conditions used to control stringency include temperature, salt concentration, concentration of organic reagents, such as formamide, and the like.

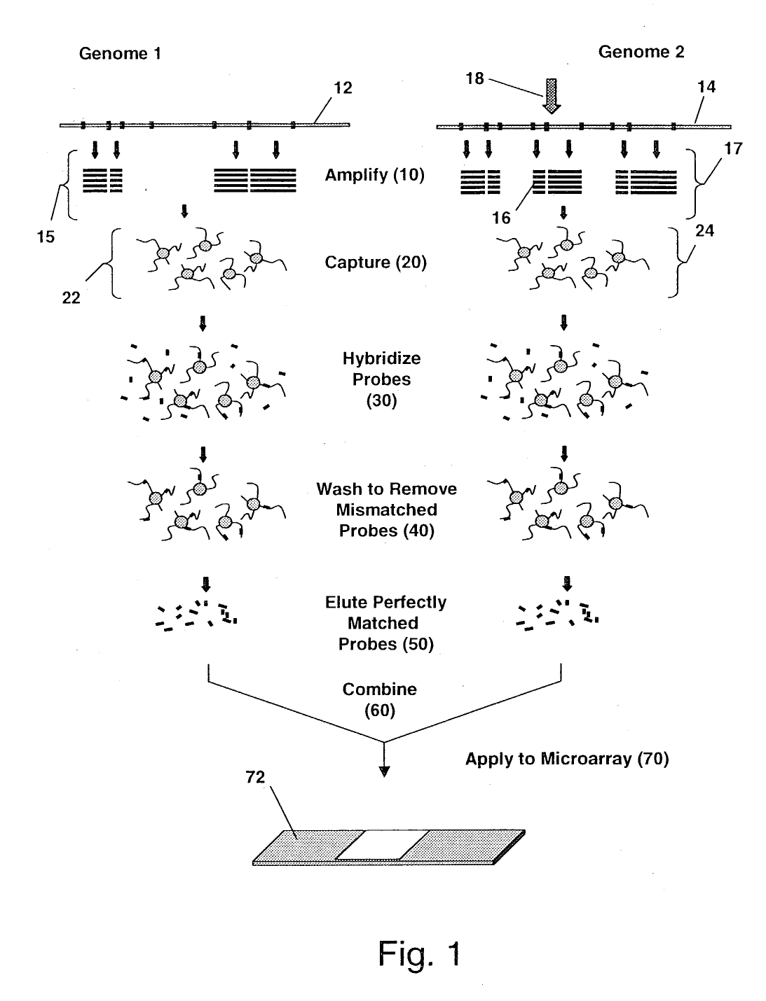

The use of isostringency probes for genetic analysis in one aspect of the invention is illustrated in FIG. 1. After separately isolating genomic DNA (12 and 14) from two individuals, the DNA is digested with one or more restriction endonucleases, e.g. r₁ and r₂, after which adaptors are ligated to the ends of the resulting fragments. The adaptors are designed to include primer binding sites so that after ligation all or a subset of the fragments can be amplified (10) in a polymerase chain reaction (PCR) to produce equivalent sets of fragments, or amplicons, for each genome. As used herein, “equivalent” in reference to populations of fragments resulting from digestion with one or more restriction endonucleases means that the genomic DNA samples are treated with the same digestion procedure and the same restriction endonucleases. Resulting fragment populations (15 and 17) may, of course, be different because of restriction site polymorphisms. Preferably, for larger genomes, e.g. greater than about 10⁸ basepairs, the complexity of the nucleic acids in the reaction can be controlled, in particular, reduced, by digesting with two or more restriction endonucleases, such as carried out in conventional amplified fragment length polymorphism (AFLP) analysis, e.g. as taught by Voss and Zabeau, U.S. Pat. No. 6,045,994, or representation difference analysis (RDA), e.g. as taught by Lisitsyn and Wigler, U.S. Pat. No. 5,436,142. The use of adaptors also permits many different fragments to amplified with the use of only a few primers in the amplification reaction.

Returning to FIG. 1, preferably, one of the two primers used to generate an amplicon includes a capture moiety, such as biotin, digoxigenin, dinitrophenol, or the like, that permits capture (20) of the amplified fragments on one or more solid phase capture supports, such as streptavidinated magnetic beads. After rendering the captured fragments single stranded, the captured amplicons (22 and 24) are separately hybridized (30) with sets of isostringency probes that differ only in the label that they carry. Probes not forming perfectly matched duplexes in the hybridization reaction are removed (40) from the captured amplicons by using highly stringent wash conditions. Isostringency probes forming perfectly matched duplexes are then eluted (50) from the captured amplicons, combined (60), and applied (70) to one or more solid phase detection supports, such as a microarray (72). Such a detection microarray contains discrete sites of complementary strands to each of the probes of the set. Isostringency probes eluted from the captured amplicons hybridize to their respective complements on the microarray where they are detected ad identified by their location on the microarray. Probes hybridizing to one amplicon, but not the other, are identified by the presence a signal generated only by label from that probe. Probes hybridizing to both amplicons ate identified by the presence of signal generated by labels from both probe sets. Probes hybridizing to neither amplicon will be substantially absent from the hybridization probes applied to the microarray, so no signal will be generated from sites where their complements are located.

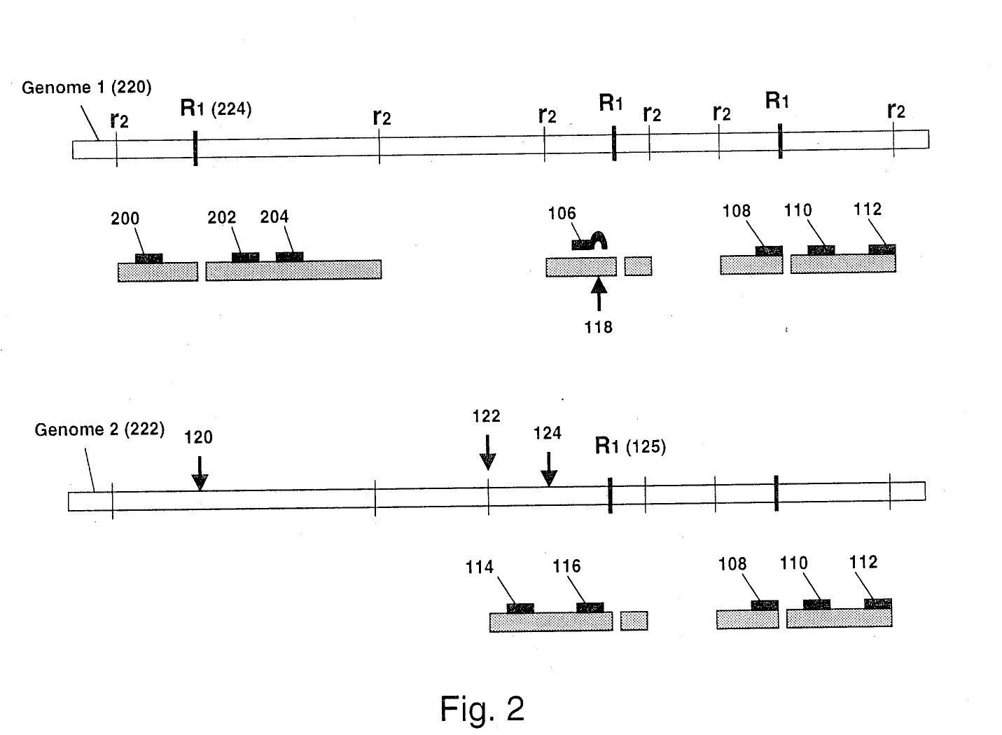

Isostringency probes hybridize to fragments amplified from different genomes as illustrated in FIG. 2. Differences in probe hybridization arise in at least two different ways. First, the occurrence of restriction site polymorphisms will cause different fragments to be present in two samples being compared. And second, the occurrence of sequence polymorphism, such as SNPs, in one of two fragments present in both samples and spanned by an isostringency probe. Referring to FIG. 2, restriction sites of restriction endonuclease R₁ and r₂ are shown in identical sections of genome 1 (220) and genome 2 (222). It is assumed for this illustration that R₁ corresponds to a “rare cutting” restriction endonuclease, for example, one that has an eight-basepair recognition site, and that r₂ corresponds to a “frequent cutting” restriction endonuclease, for example, one that has a four-basepair recognition site. Thus, essentially the only fragments amplified are those flanking the R₁ sites. If genome 2 is lacking an R₁ site at location (120), then probes 200, 202, and 204 will be absent from the probes eluted from the captured amplicon of genome 2, and their labels will make no contribution to the signals generated at their respective complementary strands on the microarray. Likewise, if the r₂ site at position (124) is absent, the fragment amplified will be defined by R₁ site (125) and r₂ site (122), which is longer than the corresponding fragment in genome 1. Thus, probe (114) will be present in the isostringency probes eluted from the captured amplicon of genome 2, but not that of genome 1. Finally, a fragment (117) of one genome may contain a sequence polymorphism (118) in a region spanned by an isostringency probe (106), such that the corresponding region of the other genome forms a perfectly matched duplex with the same isostringency probe. Probe (106) will be removed from the captured amplicon of genome 1 during the washing step, whereas the corresponding probe (116) for genome 2 will remained hybridized to its captured amplicon. Thus, signals will be generated only from the label of probe (116) at the site on the microarray containing the complements to (106) and (116).

Thus, the isostringency probes “scan” the captured amplicon of a test genome for the presence of restriction site polymorphisms that create or destroy binding sites and for the presence or absence of SNPs occurring in the binding sites of isostringency probes. In the latter circumstance, low frequency random nucleotide variation in a population will prevent a small percentage of each isostringency probe from forming perfectly matched duplexes with all target fragments from the same locus. For example, if a fragment is from a mammalian inter-genic region where the pairwise rate of nucleotide variation is about 1 per 300, then for a 12-mer isostringency probe; about 12/300 (≈4%) of the binding sites in any sample will have mismatches.

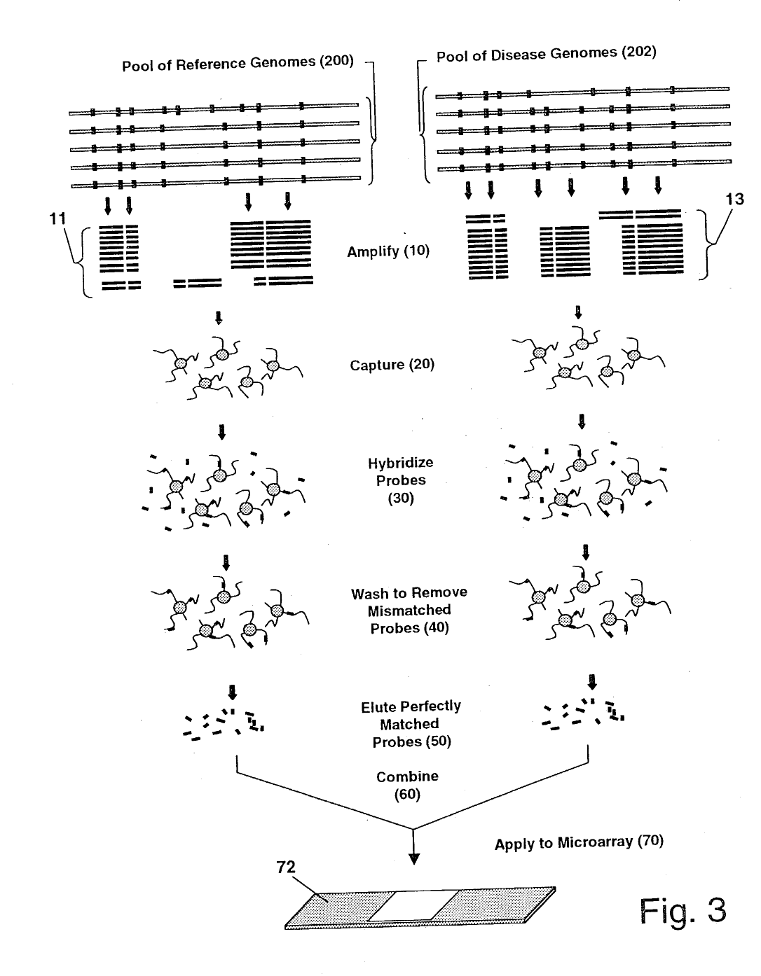

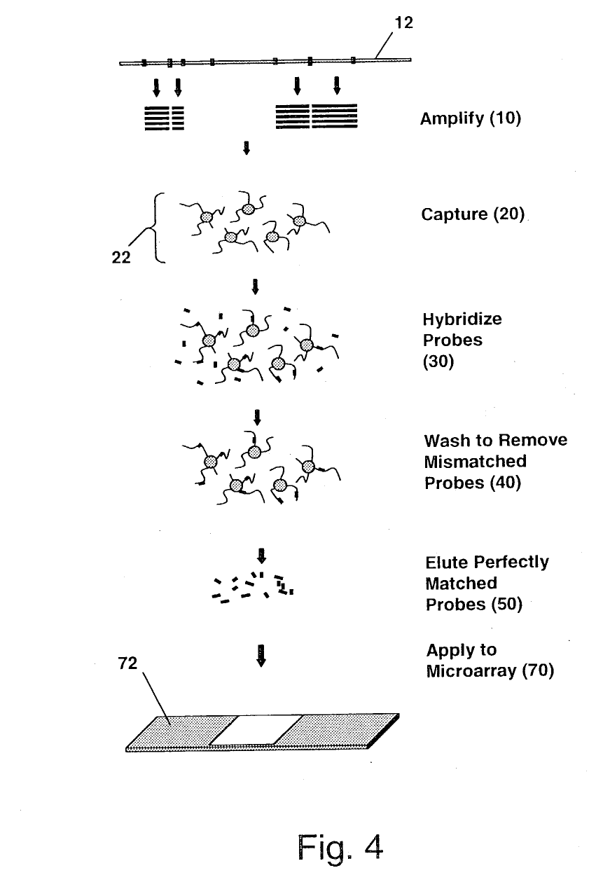

Preferably, the method of the invention is applied to pools of genomes as illustrated in FIG. 3 to determine relative frequencies of genotypes of a large plurality of loci. In this embodiment, the same procedure is followed as described for FIG. 1, except that two populations of genomes are compared in place of two individual genomes. Likewise, the method of the invention can also be applied to a single genome as shown in FIG. 4 to obtain a fingerprint, or index, of the genome. The fingerprint consists of the pattern of addresses in the microarray that accept labeled probe. This embodiment is also useful where it is desired to use a single type of label to compare relative frequencies. In this case, two genomes are compared by examining the pattern and intensity of signals generated on two identical microarrays, rather than relying on competitive hybridization of differently labeled probes to a single microarray.

The method of the invention can be applied to any type of genome to determine relative frequencies of selected sequence markers in comparison to a reference genome. As used herein, a “reference genome” is virtually any sequenced version of the genome being analyzed that allows sequence markers to be mapped along its length. A reference genome does not have to be a complete sequence of a target organism's genome. Suitable genomes for analysis include viral or bacterial genomes, and genomes of higher organisms, such as fungus, plants, and vertebrates, including birds, fish, and mammals, particularly humans and animals of economic importance, such as livestock. For larger genomes, as mentioned above, preferably the method of the invention is applied to a representation of the genome in order to reduce the complexity of the hybridization reactions. This is conveniently accomplished by amplifying a subset of restriction fragments after digestion with more than one, preferably two, restriction endonucleases. Conveniently, such digestion partitions a genome into several disjoint subsets so that the method of the invention may be applied to each of the subsets of fragments successively to obtain sequence marker frequencies at successively higher densities of loci. Alternatively, different populations of fragments can be generated by using different sets of restriction endonucleases for the digestion. Preferably, for larger genomes restriction endonuclease having a eight-basepair recognition site (“8-cutter”) is used together with a restriction endonuclease having a four-basepair recognition site (“4-cutter”). Exemplary restriction endonucleases having eight-basepair recognition sites include CciNI, FseI, NotI, PacI, SbfI, SdaI, SgfI, Sse83871I, and the like. Exemplary restriction endonucleases having four-basepair recognition sites include Tsp509I, MboI, Sau3AI, DpnII, MaeII, HpaII, MspI, BfaI, HinP1I, TaqI, MseI, MhaI, TaiI, NlaIII, ChaI, and the like. For example, in a genome of about 3×10⁹ basepairs, an 8-cutter will have about 4.6×10⁴ sites, assuming a random occurrence of the different nucleotides throughout the genome. If the genome is digested with both an 8-cutter and a 4-cutter and only fragments having one 8-cutter end and one 4-cutter end are amplified, then about 2×4.6×10⁴ fragments will be amplified for analysis. On average the fragments will be about 128 basepairs in length; thus, about 11.8 MB (=2×128×4.6×10⁴) of sequence will be amplified, or about a 0.4% sample of the genome. Polymorphisms detected by probes directed to these fragments will be uniformly distributed over the genome with an average distance about the same as the distance between the 8-cutter sites, or about 65 kilobases. This average distance can be reduced by using additional 8-cutters. For example, using NotI and TaiI and then using SbfI and Sau3A separately leads to a uniform distribution of sequence markers having an average distance of about 32 kilobases. The selection of combinations of restriction endonucleases to achieve a desired density of sequence markers and complexity of hybridization reactions in a given embodiment is a matter of design choice for one skilled in the art.

### Sequences and Composition of Isostringency Probes

Preferably, an isostringency probe of the invention comprises a label and an oligonucleotide which is capable of specifically hybridizing to a target polynucleotide by way of Watson-Crick basepairing. In reference to an isostringency probe, “length” means the number of nucleotides, or nucleotide analogs, in the oligonucleotide of the probe that can basepair with nucleotides of a target polynucleotide. Preferably, oligonucleotide probes of a given set have the same length. Oligonucleotides of the probes may comprise any nucleotide or analog thereof capable of basepairing with nucleotides of a target polynucleotide, including oligonucleotides comprising natural nucleosides linked by phosphordiester bonds, and oligonucleotides comprising analogs of either the nucleoside or the linkages connecting them. Preferably, the number of oligonucleotide probes in a set is in the range of from 500 to 40,000.

In one embodiment, the sequences of isostringency probes in any given set are selected in reference to the sequences in the amplicon generated from a reference genome. Such sequences, in turn, are determined by the restriction endonucleases selected, and perhaps, the primers used in the amplification (e.g. if primers are used to further reduce amplicon complexity, as in AFLP). Thus, for example, if a human reference genome is digested with NotI and MseI so that only NotI-MseI fragments make up the amplicon, the sequences of a set of isostringency probes are selected from complementary sequences of the same length in the approximately 11.8 megabases (MB) of sequence of the amplicon. The size of, or the number of isostringency probes in, a set is a matter of design choice which may involve trade-offs in performance and cost that are readily decided by one of ordinary skill in the art. For example, the larger the set of isostringency probes, the greater the divergence in duplex stabilities; thus, the greater the difficulty in ensuring that eluted isostringency probes are all from perfectly matched duplexes. One the other hand, a plurality of smaller-sized sets of isostringency probes may be used for each amplicon. Thus, there would be less difficulty in ensuring that eluted isostringency probes are all from perfectly matched duplexes, but at the cost of having to carry out a plurality of hybridizations instead of only one. Preferably, the lengths of isostringency probes in this embodiment are selected so that they hybridize to only one site in the amplicon. Preferably, for amplicons having a complexity of from 10 to 20 MB, isostringency probes have a length in the range of 15 to 25 nucleotides. In further preference, sequences of isostringency probes are selected so as to avoid repetitive sequence regions that may be included in an amplicon. Sequences of isostringency probes may be screened to remove those that are complementary and/or complementary at all but one or two nucleotide to common repetitive elements in human genomic DNA by publicly available software tools, such as Repeatmasker (Smit and Green, University of Washington. http://repeatmasker.genome.washington.edu). The nature and occurrence of repetitive DNA and tools for studying the phenomena are described in Smit, Current Opinion in Genetics & Development, 9: 657-663 (1999).

In another embodiment, sequences of isostringency probes are selected without reference to the sequences in an amplicon. Rather, sequences of a set of isostringency probes are selected to minimize divergence of duplex stabilities, and the length of the probes is selected so that each probe of the set hybridizes to an amplicon at a predetermined average number of sites. For example, considering the 11.8 MB amplicon described above, an 11-mer of any sequence will hybridize to such an amplicon at 2.8 sites on average (assuming the 11.8 MB can be approximated by random sequence), or a 12-mer of any sequence will hybridize to such an amplicon at 0.7 sites on average. Thus, sets of isostringency probes may be constructed that can be applied to many different amplicons. In further preference, isostringency probes in this embodiment are also selected to be minimally cross-hybridizing, e.g. as taught by Brenner et al (cited below). Briefly, the minimally cross-hybridizing property requires that the sequence of each probe in the set differ by at least two nucleotides from the sequence of every other probe in the same set. This latter property is useful when probe forming perfectly matched duplexes are isolated and applied to a microarray for detection. If the sequences of such probes differ by at least two or more nucleotides, then there will be greater specificity of duplex formation with their complements on the microarray. In this embodiment, the lengths of isostringency probes are preferably in the range of from 9 to 14 nucleotides. As above, sequences of the isostringency probes of this embodiment preferably are not complementary to repetitive sequences.

Isostringency probes comprising nucleotide analogs that confer increased duplex stability may be employed, thereby permitting the use of shorter probe lengths and/or hybridization conditions that favor the destabilization of secondary structures in target polynucleotides. Such compounds have been developed in the antisense therapeutics field and are described in the following exemplary references: Uhlman and Peyman (cited above); Crooke et al, Exp. Opin. Ther. Patents, 6: 855-870 (1996); Mesmaeker et al, Current Opinion in Structural Biology, 5: 343-355 (1995); and the like. In particular, isostringency probes of the invention may comprise stability-enhancing oligonucleotides that increase the Tm of the isostringency probes by at least an average of 1° C. per basepair over that of a probe comprising an equivalent unmodified oligonucleotide. Exemplary types of oligonucleotides for use with the invention that are capable of enhancing duplex stability include oligonucleotide N3′→P5′ phosphoramidates (referred to herein as “amidates”), peptide nucleic acids (referred to herein as “PNAs”), oligo-2′-O-alkylribonucleotides, oligonucleotides containing C-5 propynylpyrimidines, and like compounds. Such oligonucleotides are either available commercially or may be synthesized using methods described in the literature, as exemplified by the references listed in the following table:

TABLE
References disclosing methods of synthesizing
the indicated oligonucleotides

| Type of | |
|---|---|
| Oligonucleotide | References |
| Oligonucleotide-N3′→ | Letsinger et al, U.S. Pat. No. 5,476,925 |
| P5′ phosphoramidates | Gryaznov et al, U.S. Pat. No. 5,726,297 |
| (“amidates”) | Gryaznov et al, U.S. Pat. No. 5,837,835 |
| | Hirschbein et al, U.S. Pat. No. 5,824,793 |
| | [All of the above patents are hereby incorporated |
| | by reference]. |
| Peptide nucleic acids | Nielsen et al, U.S. Pat. No. 5,773,571 |
| (“PNAs”)1 | Nielsen et al, U.S. Pat. No. 5,766,855 |
| | Nielsen et al, U.S. Pat. No. 5,736,336 |
| | Nielsen et al, U.S. Pat. No. 5,714,331 |
| | Nielsen et al, U.S. Pat. No. 5,539,082 |
| | Eriksson et al, Quarterly Review of Biophysics, |
| | 29: 369-394 (1996) |
| | [All of the above references are hereby |
| | incorporated by reference]. |
| Oligo-2′-O- | Ohtsuka et al, U.S. Pat. No. 5,013,830 |
| alkylribonucleotides1 | Inoue et al, Nucleic Acids Research, 15: 6131 |
| | (1987) |
| | Shibahara et al, Nucleic Acids Research, 15: 4403 |
| | (1987) |
| | Shibahara et al, Nucleic Acids Research, 17: 239 |
| | (1989) |
| | Sproat et al, Nucleic Acids Research, 17: 3373 |
| | (1989) |
| | Sproat et al, chapter 3 in Eckstein, (cited above) |
| Oligonucleotides with | Wagner et al, Science, 260: 1510-1513 (1993) |
| C-5 | Moulds et al, Biochem., 34: 5044-5053 (1995) |
| propynylpyrimidines1 | Froehler et al, U.S. Pat. No. 5,830,653 |

Guidance for designing oligonucleotide N3′→P5′ phosphoramidates for use with the invention can be found in the patents cited above and in the following references: Gryaznov et al, Proc. Natl. Acad. Sci., 92: 5798-5802 (1995); and Chen et al, Nucleic Acids Research, 23: 2661-2668 (1995), which references are incorporated by reference. Briefly, the hybridization properties of amidates are similar to those of oligonucleotides with phosphodiester linkages (e.g. higher fraction of GC basepairs leads to a more stable duplex), except that in an amidate:RNA duplex there is an increase in Tₘ of 2.3-2.6° C. per N3′→P5′ phosphoramidate linkage, even in relatively low salt hybridization buffers, e.g. 150 mM NaCl, as compared to the more usual 0.9-1.0 M NaCl. The latter higher salt concentrations lead to duplexes of even greater stability. Labels, such as fluorescent dyes, may be conveniently attached to the 3′ amino of oligonucleotide N3′→P5′ phosphoramidates by the method disclosed in Grvaznov et al, Nucleic Acids Research, 20: 3403-3409 (1992), which is incorporated by reference. Labels may also be attached to other groups on the amidates using chemistries and reagents available for use with unmodified oligonucleotides.

Guidance for designing PNAs for use with the invention can be found in the patents and reference cited above. Briefly, in addition to the contribution to duplex stability conferred by hydrogen bonding and base stacking, the Tₘ's of PNA:RNA duplexes increase by about 1-2° C. per basepair because of the PNA backbone. The stability of PNA:RNA duplexes is almost independent of salt concentration, e.g. the Tₘ of a typical 15-mer PNA may vary no more than 5° C. over the NaCl concentration range from 10 mM to 1 M. Preferably, PNAs used with the invention consist of fewer than sixty percent purines, as a higher percentage leads to solubility problems. Preferably, PNAs used with the invention should have no more than 4-5 purines in a row and should have no more than three G's in a row. PNAs may be labeled as disclosed in Lohse et al, Bioconjugate Chem., 8: 503-509 (1997), which is incorporated by reference.

Sets containing several hundred to several thousands, or even several tens of thousands, of oligonucleotides may be synthesized directly by a variety of parallel synthesis approaches, e.g. as disclosed in Frank et al, U.S. Pat. No. 4,689,405; Frank et al, Nucleic Acids Research, 11: 4365-4377 (1983); Matson et al, Anal. Biochem., 224: 110-116 (1995); Fodor et al, International application PCT/US93/04145; Pease et al, Proc. Natl. Acad. Sci., 91: 5022-5026 (1994); Southern et al, J. Biotechnology, 35: 217-227 (1994), Brennan, International application PCT/US94/05896; Lashkari et al, Proc. Natl. Acad. Sci., 92: 7912-7915 (1995); or the like.

Isostringency probes are conveniently synthesized on an automated DNA synthesizer, e.g. an Applied Biosystems, Inc. (Foster City, Calif.) model 392 or 394 DNA/RNA Synthesizer, using standard chemistries, such as phosphoramidite chemistry, e.g. disclosed in the following references: Beaucage and Iyer, Tetrahedron, 48: 2223-2311 (1992); Molko et al, U.S. Pat. No. 4,980,460; Koster et al, U.S. Pat. No. 4,725,677; Caruthers et al, U.S. Pat. Nos. 4,415,732; 4,458,066; and 4,973,679; and the like.

As mentioned above, an important feature of the invention is the use of oligonucleotide probes comprising oligonucleotides selected from the same minimally cross-hybridizing set of oligonucleotides. In further preference, oligonucleotide probes are also selected from the same stringency class of oligonucleotides. The sequences of oligonucleotides of a minimally cross-hybridizing set differ from the sequences of every other member of the same set by at least two nucleotides. Thus, each member of such a set cannot form a duplex with the complement of any other member with less than two mismatches. The use of oligonucleotides from a minimally cross-hybridizing set increases the specificity of duplex formation between probes and probe complements. Oligonucleotides from the same stringency class form duplexes having the same stability relationship, e.g. as measured by dissociation temperature, melting temperature, or the like. Such stability relationships include having a stability measure, e.g. melting temperature, greater than or equal to a predetermined value, e.g. a melting temperature ≧40° C., or having a stability measure within a predetermined range, e.g. a melting temperature between 40° C. and 45° C. When such probes are hybridized to complementary sequences, e.g. for detection on a microarray, mismatched probes are readily removed by adjusting the stringency of the hybridization conditions so that only perfectly matched duplexes between probes of the desired stringency class and their complements remain intact and detectable.

Sequences of minimally cross-hybridizing sets of oligonucleotides may be generated by computer programs disclosed in Brenner, U.S. Pat. No. 5,604,097; Brenner et al, U.S. Pat. No. 5,846,719; and Shoemaker et al, European patent publication EP 0799897 A1; or they may be generated by random selections from large sets of artificially synthesized oligonucleotides or from fragments of natural sequences, e.g. Kaiser et al, Science, 235: 312-317 (1987); Church et al, European patent publication 0303459 A3; Matteucci et al, Nucleic Acids Research, 11: 3113-3121 (1983); Gronostajski, Nucleic Acids Research, 15: 5545-5559 (1987); and the like. Once a set is generated, further selections may be made on the set to eliminate oligonucleotides with undesired characteristics, such as too high degree of complementarity with other oligonucleotides of the same set. too many or too few G's or C's, duplex stability which places it outside of a desired stringency class, undesired distribution of mismatches, palindromic sequences, propensity to form partial duplexes with other members of the same set, and the like. Guidance for carrying out such selections is provided by published techniques for selecting optimal PCR primers and calculating duplex stabilities, e.g. Rychlik et al, Nucleic Acids Research, 17: 8543-8551 (1989) and 18: 6409-6412 (1990); Breslauer et al, Proc. Natl. Acad. Sci., 83: 3746-3750 (1986); Wetmur, Crit. Rev. Biochem. Mol. Biol., 26: 227-259 (1991); and the like.

Stringency classes may be formed by calculating a measure of duplex stability, e.g. dissociation temperature, free energy, melting temperature, or the like, then grouping oligonucleotides having similar values. For large sets of values this is conveniently accomplished using a conventional sorting algorithm, such as a standard bubble sort, Baase, Computer Algorithms (Addison-Wesley, Menlo Park, 1978). After such sorting, a stringency class may be formed by inspecting the list and selecting an appropriate subset for a particular application. For convenience, stringency classes are preferably formed with respect to similarity in dissociation temperature or melting temperature. In one embodiment, all oligonucleotides within the same stringency class have melting temperatures (or dissociation temperatures) within the same 10° C. range; preferably, such temperatures are within the same 5° C. range, and more preferably, such temperatures are within the same 2° C. range. In another embodiment, all oligonucleotides of the same stringency class have melting temperatures (or dissociation temperatures) greater than or equal to a predetermined value. Selection of such a predetermined value depends on several factors, including the number oligonucleotides desired in the set, the length of the oligonucleotides, the size of the starting set, e.g. if derived from genomic sequences, and the like. Higher stringency requirements, shorter probes, and greater number of mismatches between probes in a minimally cross-hybridizing sets lead to smaller pluralities of probes for use in accordance with the invention. Once a set of isostringency probes are selected, in embodiments where microarrays are employed for detection, the set may be conveniently tested by carrying out hybridizations of the full set to a microarray that has complements of each of the isostringency probes in the set. The hybridization and dissociation characteristics may then be observed and specific probes that do not satisfy predetermined criteria, e.g. dissociation within a predetermine wash temperature range, can be eliminated from the set.

The oligonucleotide probes of the invention can be labeled in a variety of ways, including the direct or indirect attachment of radioactive moieties, fluorescent moieties, calorimetric moieties, chemiluminescent moieties, and the like. Many comprehensive reviews of methodologies for labeling DNA and constructing DNA adaptors provide guidance applicable to constructing oligonucleotide probes of the present invention. Such reviews include Matthews et al, Anal. Biochem., Vol 169, pgs. 1-25 (1988); Haugland, Handbook of Fluorescent Probes and Research Chemicals, Sixth Edition (Molecular Probes, Inc., Eugene, 1996); Keller and Manak, DNA Probes, 2nd Edition (Stockton Press, New York, 1993); and Eckstein, editor, Oligonucleotides and Analogues: A Practical Approach (IRL Press, Oxford, 1991); Wetmur, Critical Reviews in Biochemistry and Molecular Biology, 26: 227-259 (1991); Hermanson, Bioconjugate Techniques (Academic Press, New York, 1996); and the like. Many more particular methodologies applicable to the invention are disclosed in the following sample of references: Fung et al, U.S. Pat. No. 4,757,141; Hobbs, Jr., et al U.S. Pat. No. 5,151,507; Cruickshank, U.S. Pat. No. 5,091,519: (synthesis of functionalized oligonucleotides for attachment of reporter groups); Jablonski et al, Nucleic Acids Research, 14: 6115-6128 (1986) (enzyme-oligonucleotide conjugates); Ju et al, Nature Medicine, 2: 246-249 (1996); Bawendi et al. U.S. Pat. No. 6,326,144 (derivatized fluorescent nanocrytals); Bruchez et al, U.S. Pat. No. 6,274,323 (derivatized fluorescent nanocrystals).

Preferably, one or more fluorescent dyes are used as labels for the oligonucleotide probes, e.g. as disclosed by Menchen et al, U.S. Pat. No. 5,188,934 (4,7-dichlorofluorscein dyes); Begot et al, U.S. Pat. No. 5,366,860 (spectrally resolvable rhodamine dyes); Lee et al, U.S. Pat. No. 5, 847,162 (4,7-dichlororhodamine dyes); Khanna et al, U.S. Pat. No. 4,318,846 (ether-substituted fluorescein dyes); Lee et al, U.S. Pat. No. 5,800,996 (energy transfer dyes); Lee et al, U.S. Pat. No. 5,066,580 (xanthene dyes): Mathies et al, U.S. Pat. No. 5,688,648 (energy transfer dyes); and the like. As used herein, the term “fluorescent signal generating moiety” means a signaling means which conveys information through the fluorescent absorption and/or emission properties of one or more molecules. Such fluorescent properties include fluorescence intensity, fluorescence life time, emission spectrum characteristics, energy transfer, and the like.

### Preparation of Genomic DNA and Target Polynucleotides

Virtually any population of polynucleotides may be analyzed by the method of the invention, including restriction digests, libraries of genomic fragments, cDNAs, mRNAs, or the like. Preferably, populations of polynucleotides analyzed by the invention are genomes of organisms whose sequence is known. Such genomes may be from any organism, including plant, animal, bacteria, or the like. When genomic DNA is obtained for medical or diagnostic use, it may be obtained from a wide variety of sources, including tissue biopsies, blood samples, amniotic cells, and the like. Genomic DNA is extracted from such tissues by conventional techniques, e.g. as disclosed in Berger and Kimmel, Editors, Methods in Enzymology, Vol. 152, Guide to Molecular Cloning Techniques (Academic Press, New York, 1987), or the like.

Preferably, a sample of genomic DNA is digested with at least two restriction endonucleases using conventional protocols. Preferably, one restriction endonuclease is a 7- or 8-cutter and the other restriction endonuclease is a 4-, 5-, or 6-cutter. Preferably, digestion with the selected restriction endonucleases leaves well-defined ends on the restriction fragments that each have a 2- to 4-nucleotide protruding strand to facilitate the ligation of adaptors. Adaptors are ligated to the selected subset of restriction fragments by conventional protocols, e.g. as taught in Wu et al, U.S. Pat. No. 4.617,384; Wigler et al. U.S. Pat. Nos. 6,277,606; 5,436,142; or Zabeau and Vos, U.S. Pat. No. 6,045,994, which patents are incorporated by reference. Each adaptor contains a primer binding site for carrying out preferential amplification by PCR of a selected subset of restriction fragments to produce an amplicon which is a representation of the genome. That is, the fragments make up only a few percent or less of the total genome, e.g. 1.0 percent, but they are distributed uniformly over the entire genome. Usually the selected subset of fragments consists of the fragments having one end produced by a rare-cutter, e.g. an 8-cutter, and one end produced by a frequent cutter, e.g. a 4-cutter or 6-cutter. Preferably, the sequences of the primer binding sites are substantially different so that no cross annealing occurs during an amplification reaction. In further preference, the primer selected for the adaptor ligated to the end produced by the the rare-cutter has a capture moiety. Preferably, the capture moiety is covalent attached to the 5′-end of the primer. Capture moieties include both moieties that form covalent linkage with a complementary moiety on a solid phase capture support and moieties that form non-covalent linkages with a complementary moiety on a solid phase capture support. Exemplary capture moieties forming non-covalent linkages include biotin, digoxigenin, 2,4-dinitrophenyl, peptide nucleic acid (PNA) oligomers, and the like. Exemplary capture moieties (and their complementary moieties) forming covalent linkages include, amino groups (NHS esters), amino groups (sulfonyl chloride), sulfhydryl (iodoacetyl), sulfhydryl (maleimide), and the like, e.g. Hermanson, Bioconjugate Techniques (Academic Press, New York, 1996); Nielsen, Current Opinion in Biotechnology, 12: 16-20 (2001); and like references. Preferably, biotin is used as the capture moiety. The selected restriction fragments are amplified to produce a sufficient number of replicates so that the number of binding sites for perfectly matched isostringency probes will be high enough for detection of the probes on a microarray after elution.

### Isolation and Detection of Isostringency Probes

An important feature of the invention is the separation of oligonucleotide probes forming perfectly matched duplexes with target polynucleotides from probes not forming duplexes or forming mismatched duplexes with target polynucleotides of a population being analyzed. This is accomplished by controlling the stringency of the hybridization and wash conditions.

An amplicon may be attached to a solid phase support using conventional protocols, e.g. such as described in Chiu et al, Nucleic Acids Research, 28: e31 (2000). Briefly, after ligating adaptors to the restriction digested target polynucleotide or genome, a biotinylated amplicon is created by PCR. The amplicon may be biotinylated at a 5′ end of one strand or at the 3′ end of the other strand, or it may be biotinylated at both the 5′ end of one strand and at the 3′ end of the other strand, e.g. as taught by Chiu et al (cited above). Preferably, the amplicon is biotinylated at a 5′ end of only one strand. The amplicon is capture by avidinated magnetic beads, e.g. Dynabeads M280, Dynal, Lake Success, N.Y.: New England Biolabs, Beverly, Mass.; or like product, using the manufacturer's suggested protocol in order to obtain the appropriate quantity of captured probe. (The appropriate quantity depends on the hybridization reaction volume and the desired concentration of isostringency probe. which are design parameters for one of ordinary skill in the art). After washing (e.g. 10 mM Tris-HCl, pH 7.5, 2 M NaCl, 1 mM EDTA, or like reagent), the immobilized amplicon is denatured by incubating the beads in 200 μL of 0.2 M NaOH at room temperature for 20 min, or like conditions. The supernatant is then remove to give a capture probe of single stranded amplicon. In some embodiments, two sets of single stranded capture probes are created from the same amplicon by carrying out two PCRs, one with a biotinylated forward primer and the other with a biotinylated reverse primer.

Hybridization of the isostringency probes is carried out using conventional protocols, e.g. Tijssen, ed., Laboratory Techniques in Biochemistry and Molecular Biology, Vol. 24: “Hybridization with Nucleic Acid Probes (Elsevier, N.Y., 1993). The fraction of hybridization sites on the amplicon receiving probe depends on probe concentration, which may be varied by reaction volume and quantity of probe used. Typically. a hybridization reaction contains 200 mM NaCl (or higher), 20 mM sodium citrate and a blocking agent. such as commercially available from Boehringer Mannheim, or like manufacturer. The reaction volume may be between 25 μL and 100 μL. The reaction is carried out by heating the mixture to remove any secondary structure in the amplicon, e.g. 95° C. for 5 min, followed by cooling to below, e.g. 10-20° C., the probe melting temperature over a period of about 20-30 min. Beads may then be washed several times, e.g. 2-3 times, with 70 mM sodium citrate. or like reagent, preferably at least once at the final hybridization reaction temperature and once at a temperature 2-5° C. below the melting or dissociation temperature of the isostringency probes. The perfectly matched isostringency probes may be eluted by washing with 5 μL of 50 mM ammonium hydroxide for 5 min at 80° C., or like conditions.

Preferably, the eluted isostringency probes are detected by hybridizing them to their complements on one or more solid phase supports. Such supports may take a variety of forms, e.g. particulate, single-piece and planar, such as a glass slide, and may be composed of a variety of materials, e.g. glass, plastic, silicon. polystyrene, or the like. Particulate solid phase detection supports include microspheres, particularly fluorescently labeled microspheres, e.g. Han et al, Nature Biotechnology, 19: 631-635 (2001); Kettman et al, Cytometry, 33: 234-243 (1998); and the like. Preferably, isostringency probes are detected by hybridizing them to their complementary sequences on a conventional microarray. Such microarrays may he manufactured by several alternative techniques, such as photo-lithographic optical methods, e.g. Pirrung et al, U.S. Pat. No. 5,143,854, Fodor et al, U.S. Pat. Nos. 5,800,992; 5,445,934; and 5,744,305; fluid channel-delivery methods, e.g. Southern et al, Nucleic Acids Research, 20: 1675-1678 and 1679-1684 (1992); Matson et al, U.S. Pat. No. 5,429,807, and Coassin et al, U.S. Pat. Nos. 5,583,211 and 5,554,501; spotting methods using functionalized oligonucleotides, e.g. Ghosh et al, U.S. Pat. No. 5,663,242; and Bahl et al, U.S. Pat. No. 5,215,882: droplet delivery methods, e.g. Caren et al, U.S. Pat. No. 6,323,043; Hughes et al, Nature Biotechnology, 19: 342-347 (2001); and the like. The above patents disclosing the synthesis of spatially addressable microarrays of oligonucleotides are hereby incorporated by reference. Preferably, microarrays used with the invention contain from 100 to 500,000 hybridization sites; more preferably, they contain from 200 to 250,000 hybridization sites. still more preferably, they contain from 500 to 40,000 hybridization sites; and most preferably, they contain from 500 to 25,000 hybridization sites.

Guidance for selecting conditions and materials for applying labeled oligonucleotide probes to microarrays may be found in the literature, e.g. Wetmur, Crit. Rev. Biochem. Mol. Biol. 26: 227-259 (1991); DeRisi et al. Science, 278: 680-686 (1997); Wang et al, Science, 280: 1077-1082 (1998); Duggan et al, Nature Genetics. 21: 10-14 (1999); Schena, Editor, Microarrays: A Practical Approach (LRL Press, Washington, 2000): Hughes et al (cited above); Fan et al, Genomics Research, 10: 853-860 (2000); and like references. These references are hereby incorporated by reference. Typically, application of isostringency probes to a solid phase detection support includes three steps: treatment with a pre-hybridization buffer, treatment with a hybridization buffer that includes the probes, and washing under stringent conditions. A pre-hybridization step is employed to suppress potential sites for non-specific binding of probe. Preferably, pre-hybridization and hybridization buffers have a salt concentration of between about 0.8-1.2 M and a pH between about 7.0 and 8.3. Preferably, a pre-hybridization buffer comprises one or more blocking agents such as Denhardt's solution heparin), fragmented denature salmon sperm DNA, bovine serum albumin (BSA), SDS or other detergent, and the like. An exemplary pre-hybridization buffer comprises 6×SSC (or 6×SSPE), 5× Denhardt's solution, 0.5% SDS, and 100 μg/ml denatured, fragmented salmon sperm DNA, or an equivalent defined-sequence nucleic acid. Another exemplary pre-hybridization buffer comprises 6×-SSPE-T (0.9 M NaCl 60 mM NaH2PO4, 6 mM EDTA (pH 7.4), 0.005% Triton X-100) and 0.5 mg/ml BSA. Pre-hybridization and hybridization buffers may also contain organic solvents, such as formamide to control stringency tetramethylammonium chloride to negate base-specific effects, and the like. An exemplary hybridization buffer is SSPE-T and the desired concentration of isostringency probe. After hybridization, unbound and non-specifically bound isostringency probe is removed by washing the detection support under stringent conditions. Preferably, stringency of the wash solution is controlled by temperature, organic solvent concentration, or salt concentration. More preferably, the stringency of the wash conditions are determined to be about 2-5° C. below the melting temperature of the isostringency probes at the salt concentration and pH of the wash solution. Preferably, the salt concentration of the wash solution is between about 0.01 to 0.1 M.

Instruments for measuring optical signals, especially fluorescent signals, from labeled tags hybridized to targets on a microarray are described in the following references which are incorporated by reference: Stern et al. PCT publication WO 95/22058; Resnick et al, U.S. Pat. No. 4,125,828; Karnaukhov et al, U.S. Pat. No. ,354,114; Trulson et al, U.S. Pat. No. 5,578,832; Pallas et al, PCT publication WO 98/53300; Brenner et al, Nature Biotechnology, 18: 630-634,(2000); and the like.

The following examples serve to illustrate the present invention and are not meant to be limiting. Selection of many of the reagents, e.g. enzymes, vectors, and other materials; selection of reaction conditions and protocols; and material specifications, and the like, are matters of design choice which may be made by one of ordinary skill in the art. Extensive guidance is available in the literature for applying particular protocols for a wide variety of design choices made in accordance with the invention, e.g. Sambrook et al, Molecular Cloning, Second Edition (Cold Spring Harbor Laboratory, New York, 1989); Ausubel et al, editors, Current Protocols in Molecular Biology (John Wiley & Sons, New York, 1997); and the like.

### Arbitrary Sequence 12-mer Isostringency Probes

In this example, a set of 20,000 12-mer isostringency probes sequences are generated that have dissociation temperatures in the range of 56° C. to 58° C. Dissociation temperatures were calculated using a conventional algorithm, Wetmur (cited above), and implemented by the program of Appendix I. 12-mer sequences were generated and sequences having dissociation temperatures in the desired range were selected. after which the set was filtered to remove palindromic sequences and one member of any pair capable of forming duplexes having eight or more consecutive basepairs. This resulted in a set of more than thirty thousand 12-mers. From these twenty thousand are selected. A complementary 25-mer microarray of oligonucleotides is obtained from Agilent Technologies (product number G2507A, Palo Alto, Calif.). The oligonucleotide complements on the microarray consist of oligo-dT spacers followed by the complements of the twenty thousand 12-mer isostringency probes.

APPENDIX I
Source Codes of Programs for Generating 12-mer Isostringency Probes
Having Dissociation Temperatures between 56° C. and 58° C.

Program p12tm

| c | |
|---|---|
| c | |
| c | Program p12tm calculates the tm of every 12-mer and sorts |
| c | the 12-mers having a melting temperature between 56° C. and |
| c | 58° C. tm is calculated using an algorithm from Wetmur (cited |
| c | above). |
| c | |
| c | |

dimension htable(4,4),stable(4,4)
integer*2 nbase(12)
common/history/nbase,htable,stable

| c | |
|---|---|
| c | |
| c | |
| c | |
| c | Read thermodynamic parameters. |
| c | |
| c | |

open(1,file=‘h.dat’ ,form=‘formatted’,status=‘old’)
do 100 i=1,4

| | |
|---|---|
| 100 | read(1,101)(htable(i,j),j=1,4) |
| 101 | format(4(f4.1,1x)) |
| | close (1) |

c
c

open(1,file=‘s.dat’ ,form=‘formatted’,status=‘old’)
do 150 i=1,4

| | |
|---|---|
| 150 | read(1,151)(stable(i,j),j=1,4) |
| 151 | format(4(f5.4,1x)) |
| | close (1) |

c
c
c
c
c
c

open(7,file=‘56-58.dat’,status=‘replace’)

c
c

```
  do 1000 k1=1,4
    do 1000 k2=1,4
      do 1000 k3=1,4
        do 1000 k4=1,4
          do 1000 k5=1,4
            do 1000 k6=1,4
              do 1000 k7=1,4
                do 1000 k8=1,4
                  do 1000 k9=1,4
                    do 1000 k10=1,4
```

do 1000 k11=1,4
do 1000 k12=1,4

c
c

nbase(1)=k1
nbase(2)=k2
nbase(3)=k3
nbase(4)=k4
nbase(5)=k5
nbase(6)=k6
nbase(7)=k7
nbase(8)=k8
nbase(9)=k9
nbase(10)=k10
nbase(11)=k11
nbase(12)=k12

c

call tm12(dtemp)

c

if(dtemp.ge.56.and.dtemp.le.58) then

c

write(7,133) (nbase(j),j=1,12)
endif

| c | |
|---|---|
| c | |
| 1000 | continue |
| 133 | format(12i1) |
| c | |

close (7)

| c | |
|---|---|
| | end |
| c************************************************************ | |
| c************************************************************ | |
| c | |
| | subroutine tm12 (dtemp) |
| c | |
| c | |
| | dimension htable(4,4) ,stable(4,4) |
| | integer*2 nbase(12) |
| | common/history/nbase,htable,stable |
| c | |
| c | |
| | r=.00199 |
| | conc=.000000001 |
| c | |
| c | |
| c | Calculate Tm of 12-mer |

c
c

i1=nbase(1)
i2=nbase(2)
i3=nbase(3)
i4=nbase(4)
i5=nbase(5)
i6=nbase(6)
i7=nbase(7)
i8=nbase(8)
i9=nbase(9)
i10=nbase(10)
i11=nbase(11)
i12=nbase(12)

c
c

dh=0.
ds=0.

c
c

| | |
|---|---|
| dh=dh | + htable(i1,i2)+htable(i2,i3)+htable(i3,i4) |

| | |
|---|---|
| 2 | + htable(i4,i5)+htable(i5,i6)+htable(i6,i7) |
| 3 | + htable(i7,i8)+htable(i8,i9)+htable(i9,i10) |
| 4 | + htable(i10,i11)+htable(i11,i12) |

c
c

| | |
|---|---|
| ds=ds | + stable(i1,i2)+stable(i2,i3)+stable(i3,i4) |

| | |
|---|---|
| 2 | + stable(i4,i5)+stable(i5,i6)+stable(i6,i7) |
| 3 | + stable(i7,i8)+stable(i8,i9)+stable(i9,i10) |
| 4 | + stable(i10,i11)+stable(i11,i12) |

c
c

dtemp=dh/(ds−r*log(conc)) −273.2

c
c

```
  return
  end
```

c
c

Program fpal

| c | |
|---|---|
| c | |
| c | Program fpal reads 12-mers from file 56-58.dat |
| c | and removes any 12-mers that are palindromic |
| c | |
| c | |

integer*2 nmers(1500000,12),nbase(12)

| c | |
|---|---|
| c | |
| c | Read 12-mers. |
| c | |

| | n=0 |
|---|---|
| | open(1,file=‘56-58.dat’ ,form=‘formatted’,status=‘old’) |
| 100 | continue |

n=n+1
read(1,101)(nmers(n,i),i=1,12)
if(nmers(n,1).eq.0) then

| | goto 199 |
|---|---|
| | endif |
| | goto 100 |
| 199 | close(1) |
| | write(*,198)n |
| | pause |
| 101 | format(12i1) |
| 198 | format(10x, ‘number of 12-mers=’,i8) |
| c | |

| | open(7,file=‘56-58f1.dat’,status=‘replace’) |
|---|---|
| c | |
| c | |
| | npal=0 |
| | do 1000 k=1,n |
| c | |
| c | |
| c | Test for palindromes. Note that with the nt's |
| c | coded as numbers (a=1, c=2, g=3, & t=4), perfect |
| c | matches add to 5. |
| c | |

ic1=nmers(k,1)+nmers(k,12)
ic2=nmers(k,2)+nmers(k,11)
ic3=nmers(k,3)+nmers(k,10)
ic4=nmers(k,4)+nmers(k,9)
ic5=nmers(k,5)+nmers(k,8)
ic6=nmers(k,6)+nmers(k,7)

c
c

if(ic1.eq.5.and.

| | |
|---|---|
| 1 | ic2.eq.5.and. |
| 2 | ic3.eq.5.and. |
| 3 | ic4.eq.5.and. |
| 4 | ic5.eq.5.and. |
| 5 | ic6.eq.5) then |
| | npal=npal+1 |
| | write(*,902)(nmers(k,j),j=1,12),npal |

| | |
|---|---|
| 902 | format(10x,12i1,4x,i8) |

else

| | write(7,901)(nmers(k,j),j=1,12) |
|---|---|
| 901 | format(12i1) |

endif

| c | |
|---|---|
| 1000 | continue |
| c | |
| c | |

| | |
|---|---|
| | do 2000 k=1,12 |
| 2000 | nbase(k)=0 |
| | write(7,901)(nbase(i),i=1,12) |
| c | |
| c | |
| | close(7) |
| c | |

end

c************************************************************
c************************************************************
c
c

Program dimer

| c | |
|---|---|
| c | |
| c | Program dimer reads 12-mers from file 56-58f1.dat |
| c | (output of p12-tm) and removes any 12-mers that form |
| c | dimmers with 11nt, 10nt, 9nt, or 8nt overlaps. |
| c | |
| c | |

integer*2 nmers(1500000,12),jmers(1500000,12),nbase(12)

| c | |
|---|---|
| c | Read 12-mers. |
| c | |

| | |
|---|---|
| | n=0 |
| | open(1,file=‘56-58f1.dat’ ,form=‘formatted’,status=‘old’) |
| 100 | continue |
| | n=n+1 |
| | read(1,101)(nmers(n,i),i=1,12) |
| | if(nmers(n,1).eq.0) then |

| | goto 199 |
|---|---|
| | endif |
| | goto 100 |
| 199 | close(1) |
| | write(*,198)n |
| | pause |
| 101 | format(12i1) |
| 198 | format(10x,‘number of 12-mers’,i8) |
| 902 | format(10x,i8) |
| c | |
| c | |

open(7,file=‘56-58f2.dat’,status=‘replace’)

c
c

jx=1

| c | |
|---|---|
| c | |
| | do 1050 ix=1,12 |
| | jmers(1,ix)=nmers(1,ix) |
| 1050 | continue |
| c | |
| c | |

| | |
|---|---|
| c | Test for dimer formation. Note that with the nt's |
| c | coded as numbers (a=1, c=2, g=3, & t=4), perfect |
| c | matches add to 5. |
| c | |
| c | |

do 1000 kk=1,n

do 1060 nn=1,jx

c
c

i1=jmers(nn,2)+nmers(kk,12)
i2=jmers(nn,3)+nmers(kk,11)
i3=jmers(nn,4)+nmers(kk,10)
i4=jmers(nn,5)+nmers(kk,9)
i5=jmers(nn,6)+nmers(kk,8)
i6=jmers(nn,7)+nmers(kk,7)
i7=jmers(nn,8)+nmers(kk,6)
i8=jmers(nn,9)+nmers(kk,5)
i9=jmers(nn,10)+nmers(kk,4)
i10=jmers(nn,11)+nmers(kk,3)
i11=jmers(nn,12)+nmers(kk,2)

c
c

j1=jmers(nn,3)+nmers(kk,12)
j2=jmers(nn,4)+nmers(kk,11)
j3=jmers(nn,5)+nmers(kk,10)
j4=jmers(nn,6)+nmers(kk,9)
j5=jmers(nn,7)+nmers(kk,8)
j6=jmers(nn,8)+nmers(kk,7)
j7=jmers(nn,9)+nmers(kk,6)
j8=jmers(nn,10)+nmers(kk,5)
j9=jmers(nn,11)+nmers(kk,4)
j10=jmers(nn,12)+nmers(kk,3)

c
c

k1=jmers(nn,4)+nmers(kk,12)
k2=jmers(nn,5)+nmers(kk,11)
k3=jmers(nn,6)+nmers(kk,10)
k4=jmers(nn,7)+nmers(kk,9)
k5=jmers(nn,8)+nmers(kk,8)
k6=jmers(nn,9)+nmers(kk,7)
k7=jmers(nn,10)+nmers(kk,6)
k8=jmers(nn,11)+nmers(kk,5)
k9=jmers(nn,12)+nmers(kk,4)

c
c

11=jmers(nn,5)+nmers(kk,12)
12=jmers(nn,6)+nmers(kk,11)
13=jmers(nn,7)+nmers(kk,10)
14=jmers(nn,8)+nmers(kk,9)
15=jmers(nn,9)+nmers(kk,8)
16=jmers(nn,10)+nmers(kk,7)
17=jmers(nn,11)+nmers(kk,6)
18=jmers(nn,12)+nmers(kk,5)

c
c

if((i1.eq.5.and.i2.eq.5.and.i3.eq.5.and.i4.eq.5.and.

| | |
|---|---|
| 1 | i5.eq.5.and.i6.eq.5.and.i7.eq.5.and.i8.eq.5.and. |
| 1 | i9.eq.5.and.i10.eq.5.and.i11.eq.5).or. |
| 1 | (j1.eq.5.and.j2.eq.5.and.j3.eq.5.and.j4.eq.5.and. |
| 1 | j5.eq.5.and.j6.eq.5.and.j7.eq.5.and.j8.eq.5.and. |
| 1 | j9.eq.5.and.j10.eq.5).or. |
| 1 | (k1.eq.5.and.k2.eq.5.and.k3.eq.5.and.k4.eq.5.and. |
| 1 | k5.eq.5.and.k6.eq.5.and.k7.eq.5.and.k8.eq.5.and. |
| 1 | k9.eq.5).or. |
| 1 | (11.eq.5.and.12.eq.5.and.13.eq.5.and.14.eq.5.and. |
| 1 | 15.eq.5.and.16.eq.5.and.17.eq.5.and.18.eq.5)) then |
| | goto 1000 |
| | endif |

| c | |
|---|---|
| c | |
| 1060 | continue |
| c | |
| c | |

```
  jx=jx+1
  do 1061 jz=1,12
```

jmers(jx,jz)=nmers(kk,jz)

| 1061 | continue |
|---|---|
| c | |
| c | |
| | ntest=mod(jx,10000) |
| | if(ntest.eq.0) then |
| | write(*,1400)jx |
| 1400 | format(10x,i8) |
| | endif |
| c | |
| c | |

| | |
|---|---|
| 1000 | continue |
| c | |
| c | |

do 3000 kg=1,jx

write(7,101) (jmers(kg,km),km=1,12)

| | |
|---|---|
| 3000 | continue |
| c | |
| c | |

| | |
|---|---|
| | do 4000 k=1,12 |
| 4000 | nbase(k)=0 |
| | write(7,101) (nbase(i),i=1,12) |
| c | |
| c | |
| | close(7) |
| c | |
| c | |

end

| c************************************************************ | |
|---|---|
| c************************************************************ | |
| c | |
| c | |
| | Program tag1256 |
| c | |
| c | |
| c | Program tag1256 generates a minimally cross- |

| | |
|---|---|
| c | hybridizing set of 12-mer tags from the |
| c | set 56-58f2.dat. Each 12-mer tag in the |
| c | minimally cross-hybridizing set differs from |
| c | every other member of the set by at least |
| c | 3 nt. |
| c | |
| c | |

| | |
|---|---|
| | integer*2 mset(500000,20),nbase(20),nmers(1000000,12) |
| c | |
| c | |
| | nsub=12 |
| | ndiff=3 |
| c | |

| | |
|---|---|
| c | Read 12-mers. |
| c | |

| | |
|---|---|
| | n12=0 |
| | open(1,file=‘56-58f2.dat’ ,form=‘formatted’,status=‘old’) |
| 100 | continue |
| | n12=n12+1 |
| | read(1,101) (nmers(n12,i),i=1,12) |
| | if(nmers(n12,1).eq.0) then |

| | |
|---|---|
| | goto 199 |
| | endif |
| | goto 100 |
| 199 | close(1) |
| | write(*,198)n12 |
| | pause |
| 101 | format(12i1) |
| 198 | format(10x,‘number of 12-mers=’,i8) |
| c | |
| c | |

| | |
|---|---|
| c | Choose initial tag in middle of set of 12-mers read in. |
| c | |
| c | |
| | itag=n12/2 |
| | do 200 km=1,12 |
| 200 | mset(1,km)=nmers(itag,km) |
| c | |
| c | |
| | open(7,file=‘56-58f3.dat’,status=‘replace’) |
| | jj=1 |
| c | |
| c | |
| | do 1000 kk=1,n12 |
| c | |
| c | |

nbase(1)=nmers(kk,1)
nbase(2)=nmers(kk,2)
nbase(3)=nmers(kk,3)
nbase(4)=nmers(kk,4)
nbase(5)=nmers(kk,5)
nbase(6)=nmers(kk,6)
nbase(7)=nmers(kk,7)
nbase(8)=nmers(kk,8)
nbase(9)=nmers(kk,9)
nbase(10)=nmers(kk,10)
nbase(11)=nmers(kk,11)
nbase(12)=nmers(kk,12)

c
c

The following is claimed:

1. A method of measuring frequencies of polymorphisms at multiple genetic loci, the method comprising the steps of:
amplifying a population of restriction fragments from a plurality of genomes to form an amplicon;
capturing the amplicon on one or more solid phase capture supports;
hybridizing a plurality of isostringency probes to the captured amplicons;
isolating from the captured amplicon isostringency probes that form perfectly matched duplexes with the captured amplicon; and
specifically hybridizing the perfectly matched isostringency probes with their respective complements at known locations on one or more solid phase detection supports.

2. The method of claim 1 wherein said step of amplifying further comprises the steps of: ligating adaptors to each end of said restriction fragments, each adaptor having a primer binding site; and amplifying the adaptored restriction fragments in a polymerase chain reaction using primers specific for the respective primer binding sites of the adaptors

3. The method of claim 2 wherein said step of capturing further comprises providing each of said primers specific for one of said adaptors with a capture moiety so that said amplicon produced in said polymerase chain reaction may be captured by complementary moieties on said one or more solid phase supports.

4. The method of claim 3 wherein said one or more solid phase supports is a microarray.

5. The method of claim 4 wherein said isostringency probes each have a length between 10 and 24 nucleotides and each have a melting temperature within 5° C. of every other isostringency probe.

6. A method of comparing at least two populations of polynucleotides, the method comprising the steps of:
(a) amplifying equivalent sets of restriction fragments from each population of polynucleotides to form an amplicon for each population;
(b) separately capturing each amplicon on one or more solid phase supports;
(c) hybridizing isostringency probes to each of the captured amplicons, such that isostringency probes hybridized to different amplicons have distinguishable labels;
(d) isolating from each captured amplicon isostingency probes that form perfectly matched duplexes with the captured amplicons;
(e) specifically hybridizing the perfectly matched isostringency probes with their respective complements on one or more addressable solid phase supports, so that the isostringency probes at each address generate a signal indicative of the relative frequency of their respective complements in the populations of polynucleotides.

7. A method of comparing at least two populations of polynucleotides the method comprising the steps of
(a) amplifying a representative subset of restriction fragments from each population of polynucleotides to form an amplicon for each population;
(b) separately capturing each amplicon on one or more solid phase supports;
(c) hybridizing isostringency probes to each of the captured amplicons such that isostringency probes hybridized to different amplicons have distinguishable labels;
(d) isolating from each captured amplicon isostingency probes that form perfectly matched duplexes with the captured amplicons;
(e) specifically hybridizing the perfectly matched isostringency probes with their respective complements on one or more addressable solid phase supports, so that the isostringency probes at each address generate a signal indicative of the relative frequency of their respective complements in the populations of polynucleotides.

8. A method of determining genotypes of a plurality of genetic loci uniformly distributed over a genome, the method comprising the steps of:
amplifying a population of restriction fragments from a genome to form an amplicon;
capturing the amplicon on one or more solid phase capture supports;
hybridizing a plurality of isostringency probes to the captured amplicon;
isolating from the captured amplicon isostringency probes that form perfectly matched duplexes with the captured amplicon; and
specifically hybridizing the perfectly matched isostringency probes with their respective complements at known locations on one or more solid phase detection supports.

9. A kit for detecting polymorphisms in a plurality of genes in a predetermined amplicon, the kit comprising:
one or more restriction endonucleases for generating restriction fragments of a genome;
at least two adaptors for ligating to a predetermined subset of the restriction fragments;
one or more pairs of primers for amplifying the predetermined subset of restriction fragments to produce a predetermined amplicon; and
a plurality of probes specific for the plurality of genes in the predetermined amplicon.

10. The kit of claim 10 wherein said one or more restriction endonucleases comprise a first restriction endonuclease that has a recognition site of from 6 to 8 basepairs and a second restriction endonuclease that has a recognition site of from 4 to 6 basepairs.

11. The kit of claim 11 wherein said probes are isostringency probes having a length in the range of from 10 to 24 nucleotides.

12. The kit of claim 11 wherein said first restriction endonuclease is selected from the group consisting of CciNI, FseI, NotI, PacI, SbfI, SdaI, SgfI, and Sse8387I, and said second restriction endonuclease is selected from the group consisting of Tsp509I, MboI, Sau3AI, DpnII, MaeII, HpaII, MspI, BfaI, HinP1I, TaqI, MseI, HhaI, TaiI, NlaIII, and ChaI.

13. A composition of matter consisting of a plurality of probes to a mammalian genome, the probes each having the same length in the range of from 10 to 24 nucleotides and having sequences complementary to either the sense strand or antisense strand of genes in an amplicon comprising restriction fragments produced by digestion of the mammalian genome by a first restriction endonuclease that has a recognition site of from 6 to 8 basepairs and produces restriction fragments having a protruding strand of a known sequence of at least two nucleotides and a second restriction endonuclease that has a recognition site of from 4 to 6 basepairs and produces restriction fragments having a protruding strand of a known sequence of at least two nucleotides.

14. The composition of claim 13 wherein said mammalian genome is a human genome.

15. The composition of claim 14 wherein said first restriction endonuclease is selected from the group consisting of CciNI, FseI, NotI, PacI, SbfI, SdaI, SgfI, and Sse8387I, and said second restriction endonuclease is selected from the group consisting of Tsp509I, MboI, Sau3AI, DpnII, MaeII, HpaII, MspI, BfaI, HinP1I, TaqI, MseI, HhaI, TaiI, NlaIII, and ChaI.
